(19) United States
(12) Patent Application Publication
(10) Pub. No.: US 2005/0165304 A1
(43) Pub. Date: Jul. 28, 2005

(54) Ventilation apparatus for pulmonary scinitigraphy

(76) Inventor: Roberto Albertelli, Tirrenia (IT)

Correspondence Address: DENNISON, SCHULTZ, DOUGHERTY & MACDONALD, 1727 KING STREET, SUITE 105, ALEXANDRIA, VA, 22314, US

(21) Appl. No.: 10/508,015

(22) PCT Filed: Mar. 28, 2003
(86) PCT No.: PCT/IB03/01156

(30) Foreign Application Priority Data
Mar. 28, 2002 (IT) PI2002A000018

(51) Int. Cl. A61B 6/00

(52) U.S. Cl. 600/431

(57) ABSTRACT

A ventilation apparatus (1) for pulmonary scintigraphy, having an inhalation collector (10) to which an element of inhalation (20), a feeding duct (31), defining a flow of marked aerosol (55), and an outlet duct (32), for disposal of exhaled flow and any aerosol surplus, are connected. The feeding air may be of bi-level type, whereby the air flow rate when breathing out is low, even if always positive. The collector (10) has a first interposition element (21) located at the connection with the air feeding tube (31) that only allows inlet flow towards the patient. A second interposition element (22) is located at the connection with outlet duct (32). The presence of one-way valve/s (21,22) allows to breath the marked aerosol in the bronchial branches of the patient (20) in a way completely natural following the physiologic respiration and avoiding hyper-accumulation of marker in the largest bronchial branches. Scintigraphy is allowed also during a Non Invasive Mechanical Ventilation, carried out through the ventilation apparatus (1).

### DESCRIPTION

The present invention relates to the medical field and in particular it relates to a ventilation apparatus for pulmonary scintigraphy, capable to provide a uniform distribution of a marking substance in a patient's lungs.

Furthermore, the invention relates to a method for inhalation of this substance.

### BACKGROUND OF THE INVENTION

As known, Lungs Ventilatory Scintigraphy is a technique for detecting the distribution of lungs ventilation in patients with pathology of the respiratory system such as bronchial asthma, chronic bronchitis and emphysema. It consists in ventilating into the patient an inert gas such as ¹³³Xe, ¹²⁷Xe, the ⁸¹mKr, or aerosol, usually ⁹⁹ mTc-DTPA (diethylen-triamino-pentacetic acid) or sulphide colloidal ⁹⁹ mTc, for then looking at its distribution in the lungs.

With the perfusion and ventilation systems presently in use, the patient breathes in marked aerosol, i.e. aerosol with dissolved radioactive material, through a mouthpiece connected on an end of a tube and connected at the other end to an aerosol apparatus, and breathes out the surplus marker through the same tube towards a radioactive material collecting filter. The air pressure is maintained steady when breathing in and out, so that the marker is deposited in the largest bronchial branches of the respiratory system. The radioactive material, therefore, deposits especially in the main bronchial branches, colliding against their walls, whereas it reaches the peripheral branches much slowly.

For taking a full picture with a “gamma camera” of the main and peripheral respiratory tubes the patient must ventilate them with a traditional aerosol apparatus for about 20/30 minutes. In fact, only such a long ventilation allows the radioactive substances to deposit also in the most peripheral tubes, in order to take an overall picture of the lungs.

On the other hand, for diffusing the radioactive material in the lungs, enough to provide readable distribution images of the ventilation (at least 1 Kc/sec), not only the above aerosol time is necessary (about 20/30 minutes) but also time is necessary for taking the images twice, i.e. preliminary images, immediately after counting 1 KC/sec, and deferred images, three/four hours later. This double test, in two different times, is necessary to avoid that the second image shows a radioactive accumulation in the main respiratory tubes. Such accumulation of radioactive material affects the acquisition of the first image, which since they are “unclean” cannot be read, unless comparing it with the second, for ascertaining possible pulmonary occlusions. In order to obtain “a clean image” it is necessary to wait for about four hours, so that the mucociliary system eliminates partially the accumulation of radioactive material. Notwithstanding the deferred images do not show said radioactive accumulation, however they cannot avoid to show the marker swallowed with the saliva through the oesophagus and the stomach, since scintigraphy detects it.

Another problem is that a scintigraphy test, during Non-Invasive Mechanical Ventilation with nasal mask, cannot be carried out without affecting the pressure control on which the apparatus is based and without polluting the apparatus and the environment with the exhaled marked aerosol.

### SUMMARY OF THE INVENTION

It is therefore object of the present invention to provide a ventilation apparatus for pulmonary scintigraphy that provides a uniform distribution of the marked aerosol in the bronchial branches, i.e. without hyper accumulation of marker, reducing the impact on the bronchial branches and making easier a direct peripheral distribution of the marker.

It is another object of the present invention to provide a ventilation apparatus for carrying out pulmonary scintigraphy even on not cooperating patients.

It is also object of the present invention to provide a ventilation apparatus for pulmonary scintigraphy capable of reducing the time necessary for executing a scintigraphical test.

It is a further object of the present invention to provide a ventilation apparatus for pulmonary scintigraphy for carrying out at the same time a non invasive mechanical ventilation with nasal mask, without affecting the pressure cycles on which the apparatus works and without polluting the apparatus and the environment with exhaled marked aerosol.

These and other objects are achieved by the ventilation apparatus for pulmonary scintigraphy, according to the present invention, comprising:
a feeding duct defining a flow of marked aerosol;
an outlet duct for exhaled or surplus aerosol;
an inhalation collector where said feeding and outlet ducts converge; and
a one-way interposition element movable between a first position, where it allows the flow of marked aerosol into said inhalation collector when breathing in, and a second position, where it stops or limits the flow into said inhalation collector of marked aerosol when breathing out.

The inhalation collector is located near the zone of inhalation, either through nose or mouth, to assure that the inlet marked aerosol flows through a way different from the outlet marked aerosol.

Said one-way interposition element has preferably intermediate positions in addition to an open and a closed position, in order to follow the breathing demand of the patient. In fact, when breathing in and out a gradient of pressure is produced in the inhalation collector suitable for creating a flow of marked aerosol following the physiological respiration of the patient.

Advantageously, the one-way interposition element is a flap valve.

In particular, the one-way interposition element is a flap valve formed by a stiff plastic foil that is elastically flexible.

The foil deflects only in one direction owing to an annular abutment, added to or made in the collector or in the tube, on which the foil same is connected at a side and on which it rests when in closed position. Then, each foil has a fixed portion, integral to the inner collector wall, about which the other portion of the foil can rotate between the first and the second position. The annular abutment is co-axial to the portion of the collector, or of the duct in which it is arranged, and has inner diameter smaller than that of the respective foil.

Advantageously, the flow of marked aerosol coming from a nebulizer is led into the air feeding duct upstream of the collector.

Alternatively, the flow of marked aerosol coming from a nebulizer is led directly into the collector through a duct different from that of the air feeding duct.

In a first embodiment of the invention, the inhalation collector is a three-way collector, with a central chamber and three apertures. In particular, the following are provided:
a first opening for connecting said central chamber with a feeding duct of marked aerosol;
a second opening for connecting the central chamber with an outlet duct of surplus marker connected to a radioactive material collecting filter;
a third opening for connecting the central chamber with an element of inhalation through nose or mouth;
a first flap one-way valve at the first opening for an inlet flow;
a second flap one-way valve at the second opening for an outlet flow.

More in detail, when the patient breathes in the marked aerosol the flap valve at the first opening opens completely, whereas the valve at the second opening is completely closed. When breathing out, instead, the first valve blocks partially or completely the relative aperture responsive to local pressures, reducing the flow of the aerosol, already at a low level in a bi-level ventilation, whereas the second valve opens and allows to dispose of the exhaled flow, thus following the patient's respiration.

In other words, the first valve reduces the inlet flow and allows it completely when breathing in. This creates a vortex in the large bronchial branches, without allowing the radioactive material to deposit by a stirring action. When breathing out, the surplus radioactive material is eliminated, leaving in the lungs only that necessary for marking. This way, the bronchial branches can be marked in a few minutes (from about 2 to 6 minutes) up to the final marking, and in this time it is possible to take the radiographs for looking at the transient phases.

In a second embodiment of the invention, the inhalation collector is a four-way collector, with a central chamber and four apertures. In particular, the following are provided:
a first opening for connecting said central chamber with a feeding duct of marked aerosol;
a second opening for connecting the central chamber with an outlet duct of surplus marker connected to a radioactive material collecting filter;
a third opening for connecting the central chamber with a feeding duct of air for non invasive mechanical ventilation;
a fourth opening for connecting the central chamber with an element of inhalation through nose or mouth,
a flap one-way valve at the first opening for the inlet aerosol flow.

### BRIEF DESCRIPTION OF THE DRAWINGS

Further characteristics and advantages of the present invention, will be made clearer with the following description of possible embodiments, exemplifying but not limitative, with reference to the attached drawings, wherein:

FIG. 1 shows diagrammatically a partially cross sectioned elevational side view of a first embodiment of a mouth ventilation apparatus for pulmonary scintigraphy;

FIG. 1A is a enlarged view of a detail of FIG. 1 for showing the mechanism of operation of a possible embodiment of a one-way interposition element;

FIG. 2 shows diagrammatically a perspective view of the apparatus of FIG. 1 applied to a patient;

FIG. 3 shows a perspective view of a first embodiment of an inhalation collector used in the apparatus of FIG. 1;

FIG. 4 shows the collector of FIG. 3 in a cross sectional view that shows the relative positions of the different interposition elements when breathing in;

FIG. 4A shows diagrammatically the pressure trend versus time in the apparatus of FIG. 1 when breathing in;

FIG. 5 shows the collector of FIG. 3 in a cross sectional view that shows the relative positions of the different interposition elements when breathing out;

FIG. 5A shows diagrammatically the pressure trend versus time in apparatus of FIG. 1 when breathing out;

FIG. 6 shows diagrammatically the pressure trend versus time in apparatus of FIG. 1 during a full cycle of breathing in and out;

FIG. 7 shows diagrammatically a partially cross sectioned elevational front view of a ventilation apparatus for pulmonary scintigraphy according to an embodiment alternative to that of FIG. 1 with nasal ventilation;

FIG. 8 shows a perspective view of the inhalation collector used in the apparatus of FIG. 7;

FIG. 9 shows diagrammatically a perspective view of the location of different components in the apparatus of FIG. 7, relatively to a patient when breathing in.

### DESCRIPTION OF A PREFERRED EMBODIMENT

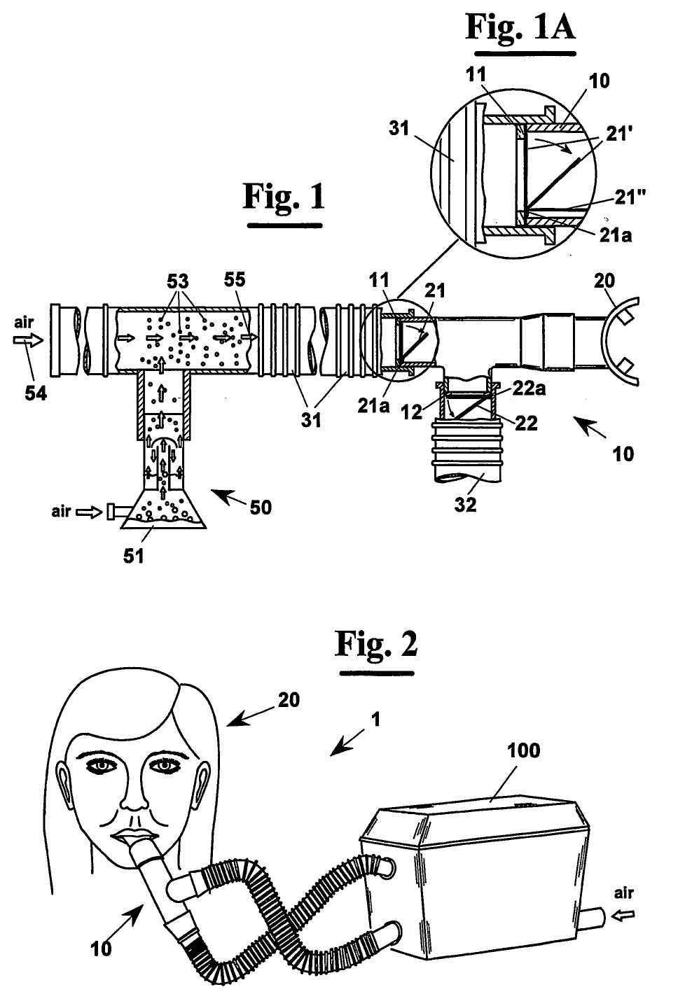

In FIG. 1 a first embodiment is shown of a ventilation apparatus 1 for pulmonary scintigraphy, according to the present invention.

It comprises an inhalation collector 10 to which an element of inhalation, for example a mouthpiece 20, a feeding duct 31, defining a flow of marked aerosol 55, and an outlet duct 32, for disposal of exhaled flow and any aerosol surplus, are connected. Normally the feeding air is of bi-level type, whereby the air flow rate when breathing out is low, even if always positive.

In particular, as shown in FIG. 1, the flow of marked aerosol 55 is obtained mixing upstream of collector 10 a current of nano-colloidal particles 53, marked for example with Tc-99m and delivered by a nebulizer 50, with an air flow 54.

As shown in FIG. 1 and in detail in FIG. 1A, collector 10 has, at the connections with ducts 31 and 32, one-way interposition elements 21 and 22. In particular, a first interposition element 21 is located at the connection with air feeding tube 31 and only allows inlet flow towards the patient, whereas a second interposition element 22 is located at the connection with outlet duct 32.

In the case shown in figures from 1 to 5, each one-way interposition element 21, or 22, is a flap valve formed by a foil of stiff thin plastic material capable of bending elastically associated to a ring abutment 11, or 12, solid or added, co-axial to the respective portion of collector 10 and having inner diameter smaller than the width of the respective valve. Each foil of plastic material 21 has a fixed portion 21a, integral to the inner wall of collector 10, about which the other portion of foil 21 can rotate between a first position 21′, where it stops or limits the flow 55 of marked aerosol, and a position 21″, wherein a maximum cross section is offered to the flow of marked aerosol towards collector 10 (FIG. 1A). Valve 22 is made in a similar way for a flow exiting from collector 10. In the closed position elements 21 and 22 rest on rings 11 and 12.

The presence of the one-way valves 21 and 22 allows to breath the marked aerosol in the bronchial branches of the patient 20 in a way completely natural following the physiologic respiration and avoiding hyper-accumulation of marker in the largest bronchial branches.

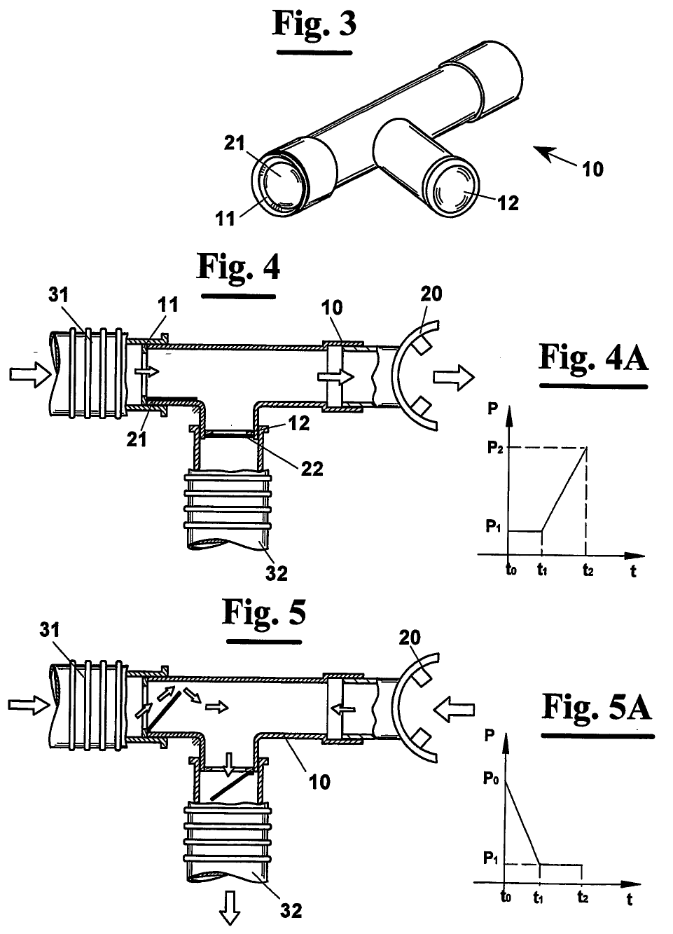

The device can be used, for example, as shown in FIG. 2, with respective ducts 31, 32 and mouthpiece 20 connected to collector 10 of FIG. 3. When breathing in, the pressure of the aerosol is maximum and the valve 21 is completely open (FIG. 4).

When breathing out, on valve 21 a certain pressure is made by the air exhaled by patient 20 in a direction opposite to the even low positive pressure of the fed aerosol flow, but of lower intensity. This causes valve 21 to move to an intermediate position that reduces but does not stop the aerosol flow into collector 10. Therefore, in collector 10 the flow of exhaled air and the surplus aerosol flow sum to each other increasing the pressure inside. This causes valve 22 to open for disposing of the surplus flow exhaled by patient 20 (FIG. 5). Valve 21, that reduces the aerosol flow towards collector 10 when breathing out and leaves it free when breathing in, when opens again produces a pressure variation that creates a vortex in the large bronchial branches of the patient without allowing the radioactive material 53 to deposit, owing to a stirring action of the vortex. When breathing out the radioactive material is eliminated along with the surplus air. This way the bronchial branches can be marked in a few minutes (from about 2 to 6 minutes).

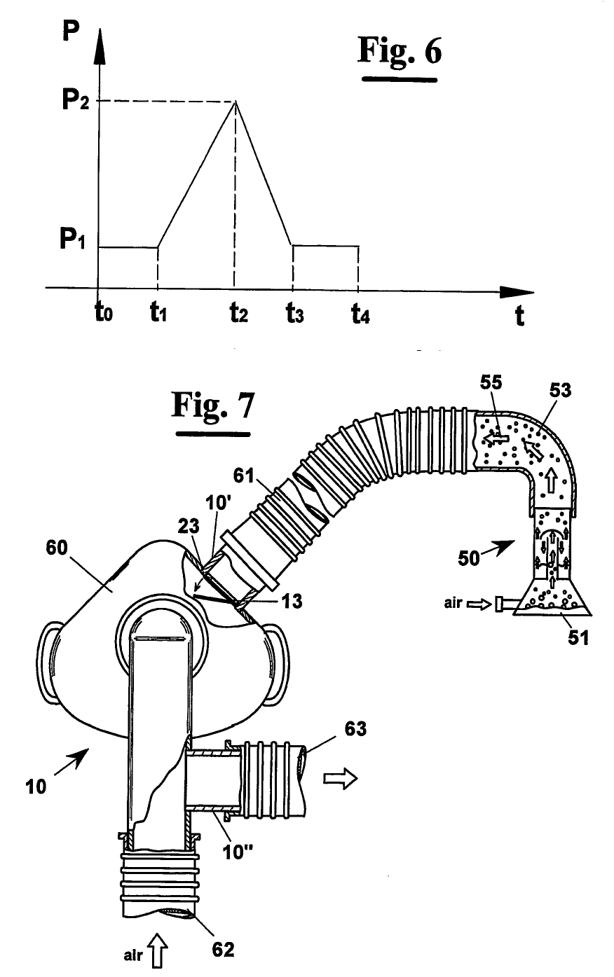

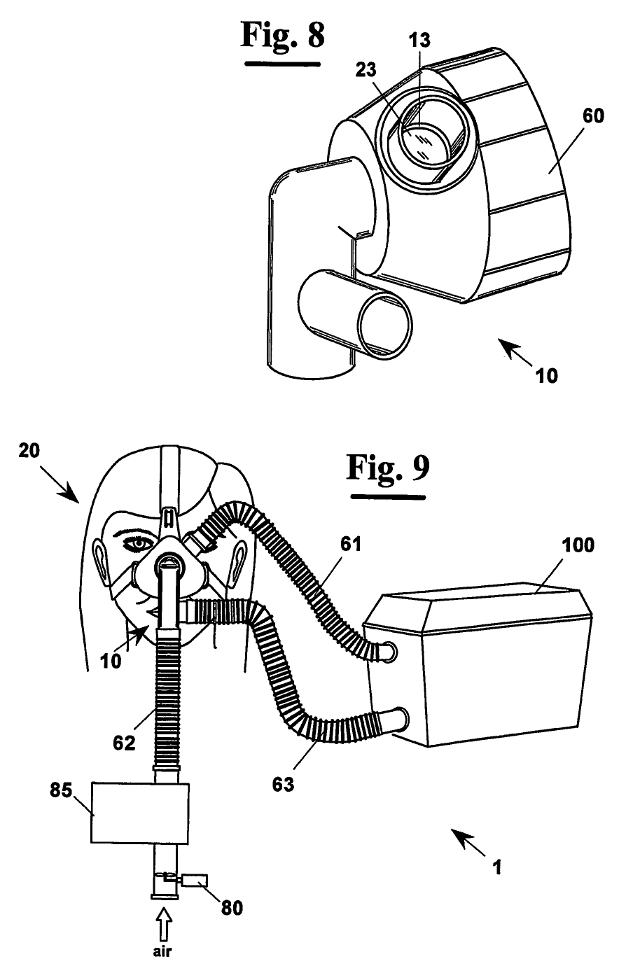

What above said is graphically shown in FIGS. 4A, 5A and 6 where qualitative trends of pressure (P) versus time (t) are indicated respectively when breathing in, when breathing out and during a full cycle of breathing in and out. In particular, when breathing in the pressure rises from a starting value P₁ up a maximum value P₂, whereas when breathing out the pressure decreases from a starting value P₀ to a value P₁<P₀, enough to keep valve 21 half open.

In figures from 7 to 9 an alternative embodiment is shown of a pulmonary ventilation scintigraphy apparatus alternative to that shown in figures from 1 to 5. This embodiment has the advantage of carrying out a pulmonary scintigraphy during a Non Invasive Mechanical Ventilation, or NIMV, without affecting the pressure cycles on which apparatus 1 works and without polluting apparatus 1 and the environment with exhaled marked aerosol. As shown in FIG. 7, a collector 10 integrated in a nasal mask 60 of an apparatus for mechanical ventilation is normally connected to a primer 80 (FIG. 9) that produces a flow of air towards the mask same.

For carrying out a pulmonary scintigraphy, using a similar apparatus, in nasal mask 60 a one-way valve 23 is arranged allowing only an inlet flow, like that above described (FIG. 1A). Valve 23 is located in collector 10 at a connection 10′ with a feeding duct 61 of an aerosol flow 55 produced by a nebulizer 50. Nasal mask 60 is, furthermore, connected by a connection 10″ of collector 10 to an outlet duct 63 of a exhalation flow containing the surplus marked aerosol, connected at the other end to a collecting filter, not shown, inserted into a lead walled box 100, in order to prevent scattering of radioactive material in the environment and to protect the operator.

During NIMV, at the moment of executing the test, it is sufficient to connect the feeding tube 61 to collector 10. In particular, nebulizer 50 works with a pressure sufficient to open valve 23 only when in the mask a vacuum is created caused by the inspiration of the patient, whereas it stops when breathing out. Differently from the previous case, the system mixes the air supplied by primer 80 and aerosol flow 55 of nano-colloidal particles 53 marked with Tc-99m coming from nebulizer 50 only in collector 10 and not before; therefore, the flow of marked aerosol to the patient 20 is adjusted by primer 80. This way, it is possible to measure the distribution of air supplied by primer 80 in the lungs of patient 20 thus allowing, with the aid of an imaging system, to check in real time the lungs apex-base ratio U/L.

The foregoing description of a specific embodiment will so fully reveal the invention according to the conceptual point of view, so that others, by applying current knowledge, will be able to modify and/or adapt for various applications such an embodiment without further research and without parting from the invention, and it is therefore to be understood that such adaptations and modifications will have to be considered as equivalent to the specific embodiment. The means and the materials to realise the different functions described herein could have a different nature without, for this reason, departing from the field of the invention. It is to be understood that the phraseology or terminology employed herein is for the purpose of description and not of limitation.

1. Ventilation apparatus for pulmonary scintigraphy comprising:
a feeding duct defining an inlet flow of marked aerosol;
an outlet duct for exhaled/surplus outlet aerosol;
an inhalation collector wherein said feeding and outlet duct converge;
a one-way interposition element movable between a first position, where it allows a flow of marked inlet aerosol when breathing in, and a second position, where it stops or limits the flow into said inhalation collector of the marked outlet aerosol when breathing out.

2. Apparatus according to claim 1, wherein said collector is located near a zone of inhalation, through nose or mouth, to assure that the inlet marked aerosol flows through a way different from the outlet marked aerosol.

3. Apparatus according to claim 1, wherein said one-way interposition element assumes intermediate positions between said first and second position to follow a demand of air of the patient.

4. Apparatus according to claim 1, wherein said one-way interposition element is a flap valve.

5. Apparatus according to claim 4, wherein said flap valve comprises a stiff plastic foil that is elastically flexible.

6. Apparatus according to claim 5, wherein said flaps deflects only one-way by an annular abutment to which said foil is connected at a side and on which it rests when in closed position.

7. Apparatus according to claim 1, wherein said inhalation collector is a three-way collector and has a central chamber with three apertures and precisely:
a first opening for connecting said central chamber with a feeding duct of marked aerosol;
a second opening for connecting the central chamber with an outlet duct of surplus marker connected to a radioactive material collecting filter;
a third opening for connecting the central chamber with an element of inhalation through nose or mouth;
a first flap one-way valve at the first opening for an inlet flow;
a second flap one-way valve at the second opening for an outlet flow.

8. Apparatus according to claim 1, wherein said inhalation collector is a four-way collector and has a central chamber with four apertures and precisely:
a first opening for connecting said central chamber with a feeding duct of marked aerosol;
a second opening for connecting the central chamber with an outlet duct of surplus marker connected to a radioactive material collecting filter;
a third opening for connecting the central chamber with a feeding duct of air for non invasive mechanical ventilation;
a fourth opening for connecting the central chamber with an element of inhalation through nose or mouth;
a flap one-way valve at the first opening for an inlet flow.

9. Inhalation collector suitable for being used in a ventilation apparatus for pulmonary scintigraphy according to claim 1.

10. Disposable kit comprising a collector, an inhalation element of through nose or mouth, ducts for inlet and outlet flows into/from said collector, suitable for being used in a ventilation apparatus for pulmonary scintigraphy according to claim 1.
